(19) United States
(12) Patent Application Publication
(10) Pub. No.: US 2002/0022858 A1
(43) Pub. Date: Feb. 21, 2002

(54) Vascular device for emboli removal having suspension strut and methods of use

(76) Inventors: Jackson F. Demond, Santa Cruz, CA (US); Amr Salahieh, Campbell, CA (US); Farhad Khosravi, San Mateo, CA (US); Jeff A. Krolik, Campbell, CA (US); Kirk Hsueh-kai Young, San Jose, CA (US); Richard J. Renati, San Jose, CA (US)

Correspondence Address: Glenn M. Seager, CROMPTON, SEAGER & TUFTE, LLC, Suite 895, Second Avenue South, Minneapolis, MN, 55401-2246, US

(21) Appl. No.: 09/764,774

(22) Filed: Jan. 16, 2001

Related U.S. Application Data
(63) Continuation-in-part of application No. 09/430,211, filed on Oct. 29, 1999.
Application No. 09/430,211 is a continuation-in-part of application No. 09/364,064, filed on Jul. 30, 1999.

(51) Int. Cl. A61M 29/00

(52) U.S. Cl. 606/200

(57) ABSTRACT

Apparatus and methods are provided for use in filtering emboli from a vessel, wherein a vascular filter disposed on a guidewire, the vascular filter comprising a support hoop disposed from a suspension strut so as to permit lateral eccentric displacement of the support hoop relative to a longitudinal axis of the guidewire. A blood permeable sac is affixed to the support hoop to form a mouth of the blood permeable sac. The support hoop is disposed obliquely relative to the guidewire and is capable of being properly used in a wide range of vessel diameters. The support hoop collapses the mouth of the blood permeable sac during removal of the vascular filter to prevent material from escaping from the sac. A delivery sheath and introducer sheath for use with the vascular filter of the present invention are also provided.

### REFERENCE TO RELATED APPLICATIONS

This application is a continuation-in-part of U.S. patent application Ser. No. 09/430,211, filed Oct. 29, 1999, which is a continuation-in-part of U.S. patent application Ser. No. 09/364,064, filed Jul.30, 1999.

### FIELD OF THE INVENTION

The present invention relates to apparatus and methods for filtering or removing matter from within a vascular system. More particularly, the present invention provides a low profile self-expanding vascular device useful for capturing emboli or foreign bodies generated during interventional procedures.

### BACKGROUND OF THE INVENTION

Percutaneous interventional procedures to treat occlusive vascular disease, such as angioplasty, atherectomy and stenting, often dislodge material from the vessel walls. This dislodged material, known as emboli, enters the bloodstream, and may be large enough to occlude smaller downstream vessels, potentially blocking blood flow to tissue. The resulting ischemia poses a serious threat to the health or life of a patient if the blockage occurs in critical tissue, such as the heart, lungs, or brain.

The deployment of stents and stent-grafts to treat vascular disease, such as aneurysms, also involves the introduction of foreign objects into the bloodstream, and also may result in the formation of clots or release of emboli. Such particulate matter, if released into the bloodstream, also may cause infarction or stroke.

Furthermore, interventional procedures may generate foreign bodies that are left within a patient's bloodstream, thereby endangering the life of the patient. Foreign bodies may include, for example, a broken guide wire, pieces of a stent, or pieces of a catheter.

Numerous previously known methods and apparatus have been proposed to reduce complications associated with embolism, release of thrombus, or foreign body material generation. U.S. Pat. No. 5,833,644 to Zadno-Azizi et al., for example, describes the use of a balloon-tipped catheter to temporarily occlude flow through a vessel from which a stenosis is to be removed. Stenotic material removed during a treatment procedure is evacuated from the vessel before the flow of blood is restored. A drawback of such previously known systems, however, is that occlusion of antegrade flow through the vessel may result in damage to the tissue normally fed by the blocked vessel.

U.S. Pat. No. 5,814,064 to Daniel et al. describes an emboli filter system having a radially expandable mesh filter disposed on the distal end of a guide wire. The filter is deployed distal to a region of stenosis, and an interventional devices, such as angioplasty balloon or stent delivery system, is advanced along the guide wire. The filter is designed to capture emboli generated during treatment of the stenosis while permitting blood to flow through the filter. Similar filter systems are described in U.S. Pat. No. 4,723,549 to Wholey et al. and U.S. Pat. No. 5,827,324 to Cassell et al.

One disadvantage of radially expandable filter systems such as described in the foregoing patents is the relative complexity of the devices, which typically comprise numerous parts. Connecting more than a minimal number of such parts to a guide wire generally increases delivery complications. The ability of the guide wire to negotiate tortuous anatomy is reduced, and the profile of the device in its delivery configuration increases. Consequently, it may be difficult or impossible to use such devices in small diameter vessels, such as are commonly found in the carotid artery and cerebral vasculature. Moreover, such filter devices are generally incapable of preventing material from escaping from the filter during the process of collapsing the filter for removal.

Umbrella-type filter systems, such as described, for example, in U.S. Pat. No. 6,152,946 to Broome et al., also present additional drawbacks. One disadvantage of such systems is that the filters have only a limited range of operating sizes. Accordingly, a number of different filters of different sizes must be available to the clinician to treat different anatomies. Still further, such filters generally do not maintain apposition to the vessel wall when blood pressure pulses pass along a vessel, e.g., due to systole. In this case, because a blood pressure pulse can cause local swelling of the vessel diameter, the pressure pulse can cause the vessel to momentarily become lifted off the perimeter of the filter, thereby permitting emboli to bypass the filter.

International Publication No. WO 98/39053describes a filter system comprising an elongated member, a radially expandable hoop and a cone-shaped basket. The hoop is affixed to the elongated member, and the cone-shaped basket is attached to the hoop and the elongated member, so that the hoop forms the mouth of the basket. The filter system includes a specially configured delivery catheter that retains the mouth of the basket in a radially retracted position during delivery.

While the filter system described in the foregoing International Publication reduces the number of components used to deploy the cone-shaped basket, as compared to the umbrella-type filter elements described hereinabove, it too has drawbacks. One such drawback is that because the hoop is fixed directly to the guide wire, the cone-shaped basket may be unable to be fully deployed in a tortuous vessel. This problem is expected to arise, for example, where the resistance of the elongated member to bend to accommodate the tortuosity of the vessel causes the hoop and basket to be lifted away from the vessel wall, thereby providing a path for emboli-laden blood to bypass the filter.

Due to the eccentric nature in the which the hoop is fastened to the elongated member in the foregoing International Application, it is expected that the perimeter of the hoop may be lifted away from the vessel wall which devices employing concentric lumens, e.g., angioplasty catheters or stent delivery systems, are brought into proximity with the filter.

Moreover, because the hoop in the aforementioned reference is directly fastened to the elongated member, there is also a risk that the basket will collapse or become wound around the elongated member due to twisting of the elongated member, e.g., during transluminal insertion of the filter, or during manipulation of the proximal end of the elongated member during insertion or withdrawal of interventional devices along the elongated member.

In view of the foregoing disadvantages of previously known apparatus and methods, it would be desirable to provide a vascular device, e.g., for use as a vascular filter, that overcomes such disadvantages and employs few components.

It would be desirable to provide a reliable vascular filter that is capable of being fully deployed in tortuous anatomy.

It also would be desirable to provide a vascular filter that is resistant to becoming disengaged from the vessel wall due to lateral movements of the guide wire to which the vascular filter is coupled.

It further would be desirable to provide a vascular filter that is capable of spanning a range of vessel sizes, thereby reducing inventory requirements.

It also would be desirable to provide a vascular filter that is resistant to becoming disengaged from the vessel wall due to local swelling of the vessel diameter as blood pressure pulses along the vessel past the filter deployment location.

It further would be desirable to provide a vascular filter that is resistant to collapse or disengagement from the vessel wall due to torsional forces applied to the guide wire to which the vascular filter is coupled.

It still further would be desirable to provide a vascular device that is capable of being contracted to a small delivery profile, thus permitting use of the device in small vessels.

### SUMMARY OF THE INVENTION

In view of the foregoing, it is an object of the present invention to provide a vascular filter that overcomes disadvantages of previously known vascular filters and foreign body removal devices, and employs few components.

It is an object of the present invention to provide a reliable vascular filter that is capable of being fully deployed in tortuous anatomy.

It is also an object of the present invention to provide a vascular filter that is resistant to becoming disengaged from the vessel wall due to lateral movements of the guide wire to which the vascular filter is coupled.

It is another object of this invention to provide a vascular filter that is capable of spanning a range of vessel sizes, thereby reducing inventory requirements.

It is a further object of the present invention to provide a vascular filter that is resistant to becoming disengaged from the vessel wall due to local swelling of the vessel diameter as blood pressure pulses along the vessel past the filter deployment location.

It is another object of the present invention to provide a vascular filter that is resistant to collapse or disengagement from the vessel wall due to torsional forces applied to the guide wire to which the vascular filter is coupled.

It is a further object of the present invention to provide a vascular device that is capable of being contracted to a small delivery profile, thus permitting use of the device in small vessels.

These and other objects of the present invention are accomplished by providing a vascular device, suitable for use as a vascular filter, that comprises a blood permeable sac affixed at its perimeter to a support hoop. In accordance with the principles of the present invention, the support hoop is attached to a distal region of an elongated member, such as a guide wire, via a suspension arrangement which permits the guide wire to rotate and move laterally relative to the support hoop, without the support hoop becoming disengaged from the vessel wall. The support hoop supports a proximally-oriented mouth of the sac when the device is deployed in a vessel. The device also may comprise a nose cone to facilitate percutaneous introduction, and a delivery sheath having one or more lumens.

In a preferred embodiment, the suspension arrangement includes a support tube disposed concentrically over the guide wire that permits the guide wire to rotate relative to the support tube without transmitting torsional forces to the filter. In addition, the support hoop includes a linear or curved flexible strut that holds the support in at a near concentric position relative to the guide wire, thereby providing the large lateral deflections of the guide wire without the guide wire contacting the support hoop.

In alternative embodiments, the suspension arrangement may further comprise additional coils formed in the flexible strut to enhance apposition of the support hoop to the vessel walls, or a nose cone mounted on the support tube. As a further alternative, the suspension arrangement may be configured as series of loops or coil turns in the guide wire proximal to the point of attachment of the support hoop, thereby isolating the filter from lateral or torsional disturbances to the proximal end of the guide wire. In still other alternative embodiments, sac bunching is mitigated by tapering the sac or attaching it to the support tube.

A single use delivery sheath and introducer sheath suitable for use with the vascular filter of the present invention are also provided, as are methods of using embodiments of the present invention.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIGS. 1A-1C are, respectively, side and ends view of an illustrative previously known vascular filter shown deployed in a straight length of vessel;

FIG. 2 is a side view of the vascular filter of FIG. 1 shown deployed in a tortuous vessel, where the stiffness of the guidewire causes the filter to partially collapse;

FIG. 3 is an side view of a vascular filter constructed in accordance with the principles of the present invention;

FIGS. 4A-4B are, respectively, side views of the vascular filter of FIG. 3 shown deployed in straight lengths of vessel of different diameters and in a tortuous vessel;

FIG. 5 is a side view illustrating that the suspension arrangement of the present invention permits torsional and lateral movement of the guide wire without displacing the support hoop or filter sac;

FIGS. 6A-6C are detailed views of the suspension arrangement and nose cone construction of the embodiment of FIG. 3, while FIG. 6C is a end view of the vascular filter taken along view line C--C of FIG. 6A;

FIGS. 7A-7C are side, top and end views of an alternative embodiment of the vascular filter of the present invention;

FIGS. 8A and 8B are side and top views of another alternative embodiment of the present invention;

FIG. 9 is a side view of a further alternative embodiment of a vascular filter of the present invention in a deployed state;

FIG. 10 is a side view of a yet another alternative embodiment of a vascular filter of the present invention in a deployed state;

FIG. 11 is detailed view of a tapered guide wire and support tube arrangement suitable for use in the present invention;

FIGS. 12A-12C are side views illustrating deployment of the vascular filter of the present invention using a single use splitable delivery sheath;

FIGS. 13A and 13B are, respectively, side and top views of an introducer sheath suitable for use with the vascular filter of the present invention; and

FIGS. 14A and 14B are side views, partially in section, illustrating use of the introducer sheath of FIG. 13 in crossing a rotating hemostatic valve.

### DETAILED DESCRIPTION OF THE INVENTION

Referring to FIGS. 1A-1C and 2, some of the disadvantages of previously known umbrella-type filters are described as context for the benefits achievable with the vascular filter of the present invention. FIG. 1A shows a previously known umbrella-type filter 10 deployed in a straight length of vessel V, with emboli E approaching with antegrade flow. Filter 10 is disposed on guidewire 12 and includes radially-extending struts 14 that support biocompatible mesh 16.

FIG. 1B illustrates a situation that may arise wherein the clinician underestimates the diameter of vessel V and deploys an undersized vascular filter 10. Because umbrella-type filters generally are capable of spanning only a narrow range of vessel diameters, the result as depicted in FIG. 1B may obtain where filter 10 is undersized for the vessel diameter. In this case, emboli E will bypass around the edges of the filter 10. Where umbrella-type filters of the kind depicted in FIG. 1 are used, the clinician must therefore exercise great care in selecting the appropriate filter size, and the hospital must carry a range of sizes to fit different patient anatomies.

Moreover, even where the clinician has selected a vascular filter appropriate for the nominal diameter of vessel V, bypass of emboli may still arise. This may occur, for example, where the vessel is subject to localized swelling as blood vessel pulses, e.g., during systole, pass along the length of the vessel. In this case, which has been observed to occur, for example, in the carotid arteries, the vessel wall may be momentarily lifted away from the perimeter of the vascular filter 10, permitting a bypass situation similar to that depicted in FIG. 1B to occur.

FIG. 1B depicts the situation that may obtain where the clinician overestimates the diameter of the vessel V, and selects filter 10 having a deployed diameter larger than the nominal vessel diameter. As illustrated in FIG. 1C, because struts 14 contact the interior surface of the vessel before becoming fully deployed, filter mesh 16 may be incompletely brought into apposition with the vessel wall around its circumference. Consequently, as depicted in FIG. 1C, folds may occur in filter mesh 16 that permit emboli E to once again bypass the filter, providing inadequate protection against embolization.

Referring now to FIG. 2, an alternative drawback of the previously known vascular filters is described, which drawback is common to both umbrella-type and single fixed hoop type disclosed in the aforementioned International Publication WO 98/39053. This problem is manifests where vascular filter 10 is inserted into tortuous anatomy, and in particular, where it is necessary to place the filter in or near curved vessel V′, such as in smaller coronary arteries and the renal arteries.

As depicted in FIG. 2, guidewire 12 on which vascular filter 10 is disposed spans the bend in vessel V′. Due to the stiffness of guidewire 12 relative to strut 14 of filter 10, when inserted in vessel bend having a small radius of curvature, strut 14 may become compressed against the inner bend surface of vessel V′. This load may in turn prevent filter 10 from fully opening (or partially collapse the effected strut), permitting emboli to bypass the filter at the outer side of the bend.

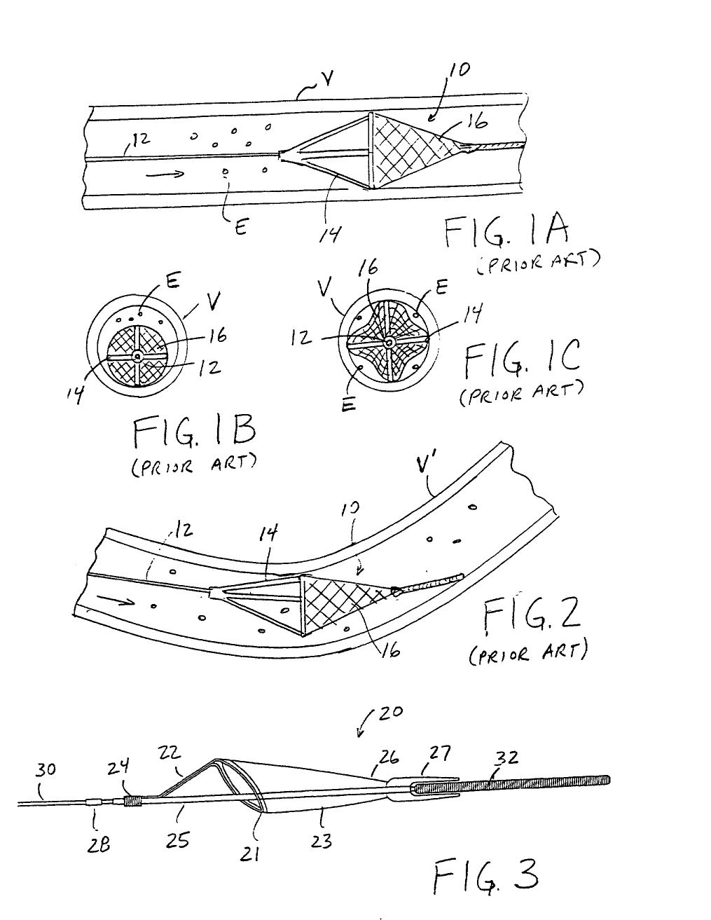

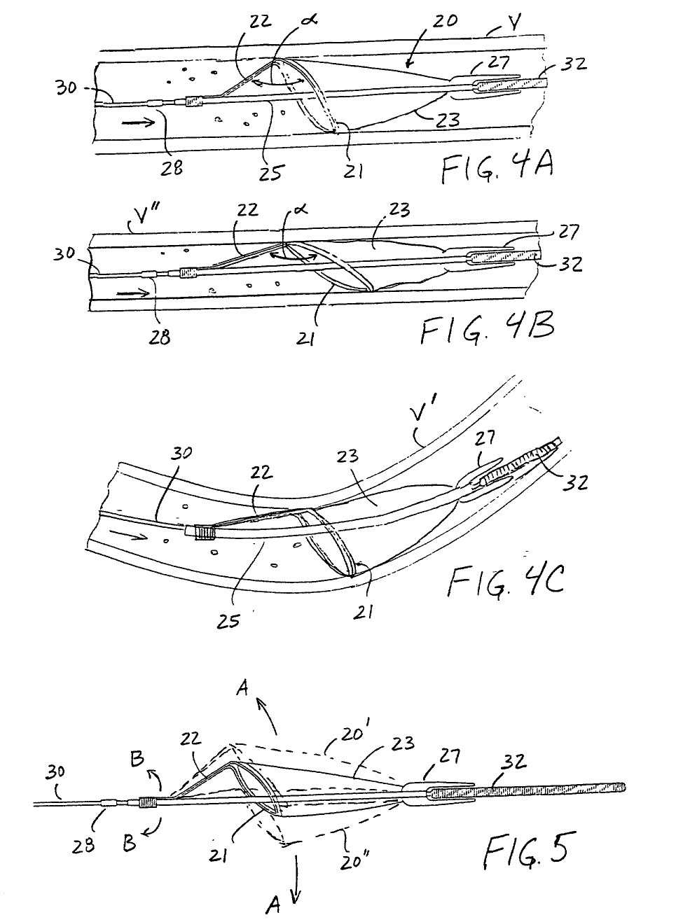

Referring now to FIG. 3, illustrative vascular filter 20 of the present invention is described. Filter 20 solves the above-described disadvantages by providing a filter that is expected to maintain apposition to a vessel wall even when used in tortuous vessels, vessels of uncertain size and those subject to localized temporal swelling caused by pressure pulsations.

Filter 20 preferably includes self-expanding support hoop 21 mounted on suspension strut 22, and supports blood permeable sac 23. Blood permeable sac comprises a biocompatible polymeric material having a multiplicity pores. Suspension strut 22 is affixed at proximal end 24 to tube 25. Distal end 26 of blood permeable sac 23 is illustratively mounted to nose cone 27, which is in turn mounted to tube 25. Filter 20 is mounted on guidewire 30 between proximal stop 28 and enlarged floppy tip 32 of the guidewire, which functions as a distal stop. Tube 25 permits guidewire 30 to rotate independently of filter 20, thereby permitting the floppy tip 32 of guidewire to be directed within the vessel without causing the blood permeable sac to become wrapped around guidewire 30.

In accordance with the principles of the present invention, suspension strut 22 positions support hoop 21 approximately concentric to tube 25 when disposed in a substantially straight length of vessel, as depicted in FIG. 4A, but permits the support hoop to become eccentrically displaced relative to support tube 25 when the filter is deployed in a curved vessel, as depicted in FIG. 4C. Thus, unlike the case described above with respect to FIG. 2, the relative differences in stiffness between guidewire 30 and suspension strut 22 facilitate, rather than impede, proper deployment of the filter 20 by permitting support hoop 22 to become eccentrically displaced relative to guidewire 30.

Referring now to FIGS. 4A and 4B, a principle advantage of the vascular filter of the present invention is described. As depicted in FIG. 4A, support hoop 21 is disposed obliquely, rather than radially, relative to the longitudinal axis of the vessel. Importantly, this arrangement permits support hoop 21 to be properly used in a variety of vessel sizes.

In larger diameter vessels, as depicted in FIG. 4A, angle α formed between suspension strut 22 and support hoop becomes less oblique, and the support hoop less elongated (more nearly perpendicular to the vessel axis). By comparison, in the smaller diameter vessel depicted in FIG. 4B, angle α becomes more oblique, and the support hoop becomes more elongated and more closely parallel to the axis of the vessel. Filter 20 has been observed to retain adequate engagement with the vessel wall around the filter circumference over a wide range of vessel sizes. Accordingly, filter 20 may properly be used in a much wider range of vessel sizes than an umbrella-type filters, while providing superior apposition to the vessel walls. Thus, for example, a filter having a nominal diameter of 6 mm may be used in vessels having diameters between about 2.5 and 6.0 mm.

Referring now to FIGS. 4B and 5, the use of single flexible suspension strut 22 permits the vascular filter to achieve good apposition to the vessel wall even in curved vessels, such as vessel V′. As shown in FIG. 5, vascular filter 20 is capable of a wide range of eccentric lateral displacements in the direction shown by arrows A (indicated by dotted lines 20′ and 20″). In addition, tube 25 permits guidewire 30 to rotate freely within the filter (shown by arrows B) without causing blood permeable sac 23 to become wrapped around the guidewire. In addition, suspension strut 22 absorbs minor longitudinal movements of guidewire 30, without causing the support hoop 21 to lose apposition to the vessel wall. Thus, transmission of minor longitudinal movements to the filter, e.g., associated with catheter exchange, are mitigated.

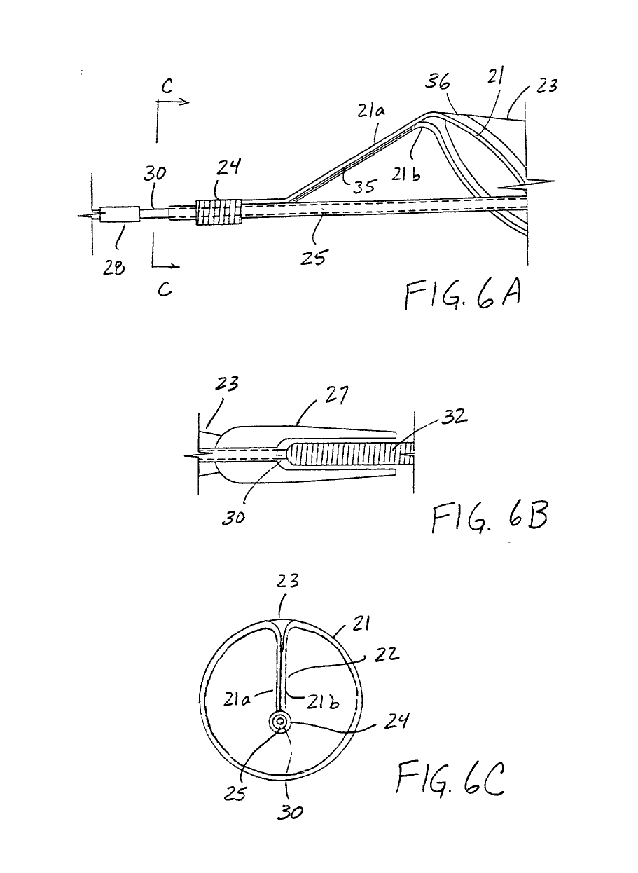

Referring now to FIGS. 6A to 6C, construction details of a preferred illustrative embodiment of the present invention are described. In FIG. 6A, detail of a preferred embodiment of support hoop 21 and suspension strut 22 are described. Suspension strut 22 preferably is formed from proximally extending portions 21a and 21b of support hoop 21, and may also include additional support member 35 welded or bonded to portions 21a and 21b. Proximal portions 21a and 21bare attached at end 24 to tube 25, for example, by wrapping, welding, crimping or other suitable bonding method. Stop 28 may comprise a weld bead, length of shrink tube, step in guidewire 30, or similar structure that limits proximal movement of tube 25 over guidewire 30.

Support hoop 21 comprises a hoop having a circular or rectangular cross-section that is formed of a super-elastic material, such as a nickel-titanium alloy (“nitinol”). During deployment and retrieval of vascular filter 20, support hoop 21 preferably folds in half and collapses to fit within the guidewire lumen of a standard balloon catheter, alternatively, a separate retrieval sheath may be employed. When vascular device 20 is in a deployed state, as depicted in FIG. 3, support hoop 21 resumes its pre-formed shape. Support hoop 21 preferably comprises nitinol wire, although it may also be formed from a multi-strand nitinol cable, a spring tempered stainless steel, or other super-elastic material.

Support hoop 21 optionally may include any of the articulation regions described in commonly owned U.S. Pat. No. 6,129,739, which is incorporated herein by reference. Thus, for example, support hoop may comprise a wire of uniform thickness, a wire having one or more reduced thickness regions, a wire having a gradual taper from its proximal ends towards its mid-point, or a pair of spines spanned by a polymer bridge or bridged by the overlapping seam of blood permeable sac 23, as described in the above-incorporated patent.

Sac 23 preferably is constructed of a thin, flexible biocompatible material, and is bonded to support hoop 21 by seam 36, or other suitable means described in the above-incorporated patent. Suitable materials for use in constructing sac 23 include polyethylene, polypropylene, polyurethane, polyester, polyethylene tetraphlalate, nylon, polytetrafluoroethylene, or combinations thereof. The sac material preferably is sufficiently thin that the sac is non-thrombogenic, and includes openings or pores that permit blood cells to pass through the sac substantially unhindered, while capturing any larger emboli, thrombus, or foreign bodies that may be released during a procedure, such as angioplasty or stent placement.

Advantageously, the number and distribution of pores may be tailored to the specification application of the vascular filter. Thus, for example, where the filter is to be used in conjunction with angioplasty of saphenous vein grafts, where large quantities of friable plaque are expected to be liberated, larger pores my be used to permit smaller particles to pass through the filter. In this case, it may be more desirable to permit small particles to pass through sac 23, rather than clog the pores interrupt blood flow. By comparison, smaller pores may be used in filters intended for carotid angioplasty applications, because less material is expected to be liberated and there may be a premium on preventing even small particle from reaching the brain.

In one preferred embodiment, sac 23 has openings or pores in a range of about 20 to 400 microns in diameter, and more preferably, about approximately 80 microns. These pore sizes permit blood cells (which have a diameter of approximately 5 or 40 microns) to easily pass through the sac, while capturing thrombus or emboli. Other pore numbers and sizes may be empirically selected with regard to the potential trade-offs in efficacy, ease of use, and other related factors that will be apparent to one of skill in the art.

Additionally, the filter membrane may be coated with coated with a lubricious coating that incorporates anti-thrombogenic agents, such as heparin. The lubricious coating, such as a hydrophobic or hydrophilic thin layer, however, should not occlude pores of the filter sac. Advantageously, such a lubricious coating may decrease friction between the blood permeable sac and the delivery sheath to enable a lower delivery profile for the vascular filter. The anti-thrombogenic agents also will reduce the amount of clot that forms on the filter membrane.

In a preferred method of manufacture, the pores in blood permeable sac 23 are formed using a laser drilling process. In this process a thin sheet of the flexible biocompatible material is first thermoformed to create sac 23, for example, by stretching the sheet over a mandrel, by dip forming, or by blow molding. Sac 23 may alternatively be fabricated from an extruded tube of the biocompatible material. A flat metal mask, having holes approximately the size of the desired pores is then used to shield the sac, and a laser having a beam diameter equal to or greater than the diameter of the material illuminates the mask. Rays of the laser beam thereby pass through the holes in the mask and strike the material to form the pores.

Laser drilling also may be accomplished using a laser having a beam diameter approximately the size of the desired pores, in which case the pores are drilled individually. Sac 23 alternatively may comprise a woven material, for example, formed from the above-mentioned polymers, having a pore diameter determined as a function of the pattern and tightness of the weave.

Referring now to FIG. 6B, nose cone 27 preferably is disposed from a distal end of tube 25, an includes an internal bore that accepts a proximal portion of floppy tip 32. This configuration shortens the overall length of floppy tip 32 extending beyond the distal end of sac 23, and may be especially desirable for filters intended in short or very tortuous vessels, such as the renal arteries. While in the embodiment of FIGS. 3-6, blood permeable sac is attached at its distal end to nose cone 27, it is to be understood that the distal end of sac 23 alternatively may be affixed to tube 25

FIG. 6C provides an end view of vascular filter 20 taken along view line C--C of FIG. 6A. Suspension strut 22 includes proximally extending portions 21a and 21b of support hoop 21, and additional support member 35 is obscured from view. Portions 21aand 21b are wrapped around tube 25 to from attachment point 24. When viewed along line C--C, support hoop 21 (and deployed in a vessel), support hoop 21 and sac 23 conform to the perimeter of the vessel, and appear circular.

In one preferred embodiment of vascular filter 20 of the present invention, filter 20 easily fits within a delivery sheath having an inner diameter of 0.033″, and, more preferably, may be used with a delivery sheath having an inner diameter of about 0.026″. The deployed diameter of support hoop 21 preferably is approximately 7 mm, while guide wire 30 preferably has a diameter of 0.014″.

Support hoop 21 preferably is constructed of 0.0035″ nitinol wire tapered (by a grinding, chemical etching, or electroless polishing process) to 0.002″ at a point on the support hoop opposite to the point where the support hoop joins suspension strut 22. Support hoop 21 also may include radiopaque features, such as gold or platinum bands (not shown), spaced at intervals around the circumference of support hoop 21, or a flat or round coil of radiopaque material wrapped around the support hoop, or a gold plated coating.

Advantageously, the compliant design of vascular filter 20 permits the filter to be contracted to its delivery state within the guide wire lumen of conventional previously known interventional devices. Accordingly, unlike previously known vascular filters, which typically require removal of the interventional device followed by re-insertion of a specially designed catheter to retrieve the vascular device, the system of the present invention reduces the time, effort and trauma of this additional step. Instead, the vascular device may be readily closed and retrieved upon completion of the interventional procedure.

It is contemplated that in operation, the vascular filter of the present invention will be deployed in a vessel using a delivery sheath, such as described hereinafter. The guidewire to which the vascular filter is attached then is used to insert an interventional device, e.g., an angioplasty catheter, atherectomy device or stent delivery system, to perform the desired diagnostic or therapeutic procedure. Upon completion of the procedure, the interventional device is advanced to capture the filter, and the vascular filter and interventional device are withdrawn together.

Alternatively, the interventional device may be held stationary, and the guidewire retracted proximally to pull the vascular filter into the guidewire lumen of the interventional device. This latter method of retrieving the vascular filter may be particularly advantageous, because as the filter is dragged along the vessel wall (or through the interior of a stent, if deployed), additional emboli material may be collected from the vessel wall. Accordingly, emboli that might not be liberated until full flow is restored to the vessel may be collected in this manner prior to closure and withdrawal of the vascular filter.

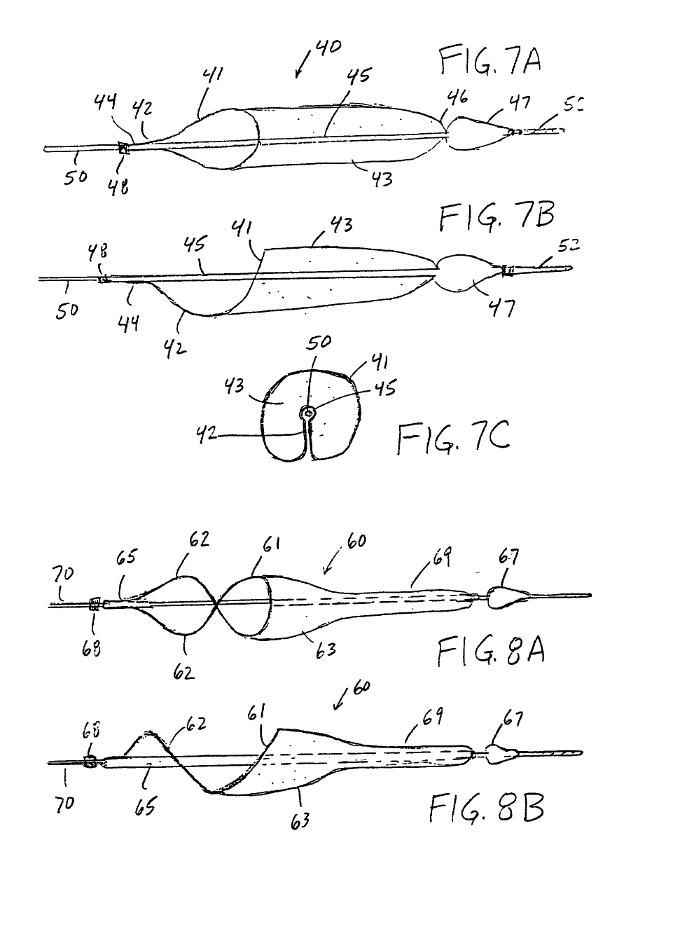

Referring now to FIGS. 7A-7C, an alternative embodiment of the vascular filter of the present invention is described. Vascular filter 40 is similar in construction to filter 20 to FIGS. 3-6, and includes support hoop 41, suspension strut 42, sac 43, fixation point 44, tube 45 and nose cone 47. Tube 45 is mounted for rotation on guidewire 50 between proximal stop 48 and floppy tip 52. Filter 40 preferably is constructed in the manner and with the materials described hereinabove.

Filter 40 differs from filter 20, described hereinabove, in that suspension strut 42 is gradually curved, and the distal end 46 of blood permeable sac 43 is affixed to tube 25, rather than nose cone 46. As for the embodiment of FIGS. 3-6, support hoop is elliptical when viewed in profile, but includes a single multi-strand suspension strut 42 that permits the filter sac to become eccentrically displaced from guidewire 50 without losing proper apposition to the vessel wall.

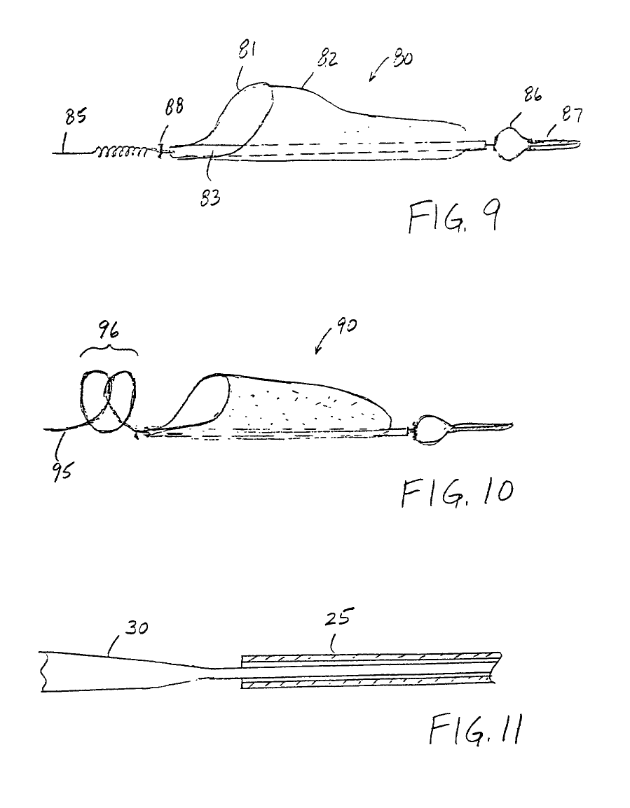

With respect to FIGS. 8A and 8B, another alternative embodiment of the vascular filter of the present invention is described. Vascular filter 60, shown in the deployed state, comprises support hoop 61 coupled to multi-turn helical suspension struts 62. Suspension struts 62 are coupled to tube 65, which is captured on guidewire 70 between proximal stop 68 and nose cone 67. Nose cone 67 is affixed to guidewire 70 distal of tube 65. The proximal end of blood permeable sac 63 is affixed to support hoop 61, while the distal end is affixed directly to tube 65. Suspension strut 62 includes one or more side turns 69 that join support hoop 61. Blood permeable sac 63 includes tapered distal portion which is expected to reduce the risk of bunching during retrieval.

In accordance with this aspect of the present invention, vascular filter 60 may be contracted to small profile delivery state. When deployed from a delivery catheter, side turns 69 expand into contact with the walls of the vessel proximal to the location at which support hoop 61 contacts the vessel wall. Side turns 69 of suspension struts 62 are expected to stabilize support hoop 61 and sac 63 when vascular filter 60 is deployed within a blood vessel. In addition, side turns 69 are expected to facilitate eccentric displacement of support hoop 61 and sac 63 relative to the longitudinal axis of a vessel. Accordingly, side turns 69 of suspension struts 62 are expected to enhance apposition of the filter against the vessel wall, and thus further enhance the safety and reliability of the device.

Referring now to FIGS. 9 and 10, additional alternative embodiments of the vascular filter of the present invention are described. In FIG. 9, vascular filter 80 comprises support hoop 81 and tapered blood permeable sac 82 mounted on tube 83. Support hoop 81 is coupled directly to the proximal end of tube 83. Filter 80 is captured on guidewire 85 between nose cone 86, which is affixed to guidewire 85 just proximal of floppy tip 87, and proximal stop 88.

In accordance with the principles of the present invention, guide wire 85 includes articulation region 89 comprising a series of small diameter coil turns. Articulation region 89 acts as a bend point in the guide wire, thereby permitting better conformance of the guidewire to tortuous anatomy and improved capture efficiency in tortuous vessels, such as illustrated in FIG. 2. Articulation region 89 therefore provides an alternative configuration for permitting the vascular filter to become displaced eccentrically displaced relative to the axis of guidewire 85.

FIG. 10 depicts an alternative configuration of the vascular filter of FIG. 9, in which filter 90 is essentially constructed in the same manner as filter 80. In this embodiment, however, guidewire 95 includes an articulation region 96 that comprises two or more large diameter coils. In addition to providing region that permits articulation of the filter relative to the axis of guidewire 95, the large diameter coils of the articulation region 96 also may assist in stabilizing the filter within the vessel after deployment.

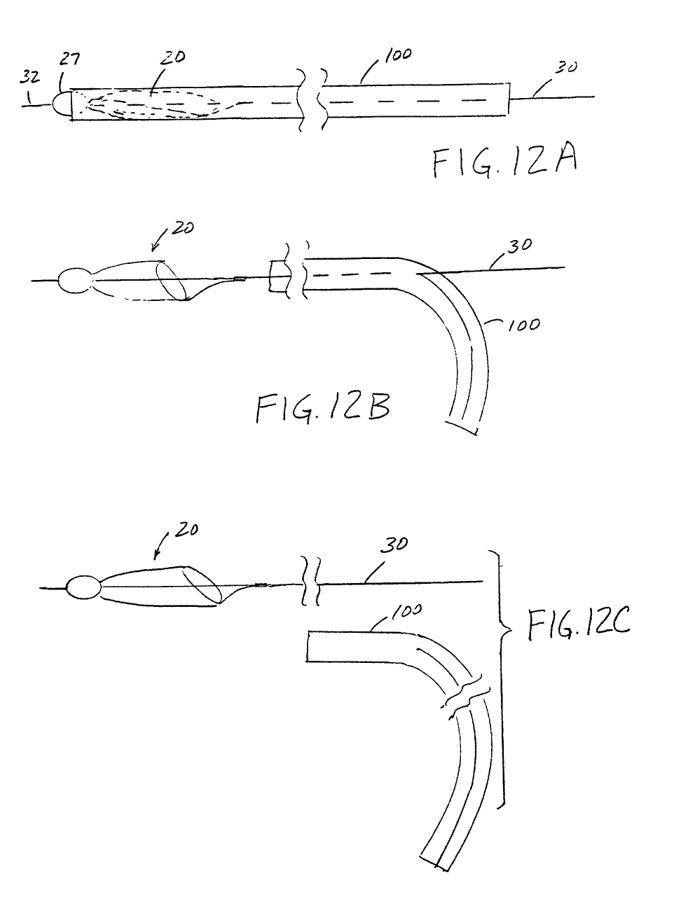

Referring now to FIG. 11, an additional feature that may be advantageously incorporated in the embodiments of the vascular filters of the present invention is described. FIG. 11 depicts an alternative configuration for the junction between a guidewire and the tube on which the filter is mounted. For example, the guidewire in FIG. 11 may be guidewire 30 of the embodiment of FIG. 3, and the tube may represent tube 25 of that embodiment. In accordance with this aspect of the present invention, guidewire 30 is tapered as shown (or includes a step, not shown) to accept tube 25. Consequently, the outer diameter of tube 25 may be made approximately the same as the guidewire thickness itself.

Because the delivery profile of the vascular filter is determined in part by the cumulative thicknesses of the components that lie adjacent to one another in the delivery sheath, use of a tapered or stepped distal region of the guidewire to accept tube 25 may enable the manufacture of significantly smaller profile devices than heretofore available. For example, in an umbrella-type filter, the delivery profile is limited by the need to have multiple struts disposed about the guidewire, and accounts for the difficulty that has been encountered in the field in constructing such filters at small delivery profiles. By comparison, a filter of the type described hereinabove when collapsed to its delivery profiled, and using the feature illustrated in FIG. 11, need not be much larger than diameter of the guidewire itself.

Referring now to FIGS. 12A-12C, a single-use delivery sheath suitable for use with the vascular filter of the present invention is described. In accordance with this aspect of the present invention, guidewire 30 may be of a length suitable for use with rapid-exchange interventional devices. Vascular filter 20 is disposed in delivery sheath 100 in its contracted configuration, with the proximal end of guidewire 30 extending from the proximal end of sheath 100 and nose cone 27 and floppy tip 32 extending from the distal end of the sheath, as shown in FIG. 12A. Delivery sheath 100 preferably comprises a soft, flexible biocompatible material, such as polyethylene or other materials typically used in catheter construction.

In accordance with known techniques, the distal region of guidewire 30 and vascular filter are percutaneously and transluminally inserted into a patient until the vascular filter is at a desired deployment site, as determined, for example, by fluoroscopy. Delivery sheath 100 is then split, either using a suitable cutting device or along a perforation seam, and retracted proximally to deploy vascular filter 20 within the vessel, as shown in FIG. 12B.

Delivery sheath 100 then is retracted proximally, with the clinician holding the proximal end of guidewire 30 in one hand, and splitting the delivery sheath along the perforation line (or with a cutting tool, not shown) until proximal end of the delivery sheath is withdrawn from the patient. At this point, the clinician may then slip the proximal end of the guidewire through the remaining unsplit portion of the delivery sheath, thereby fully removing the delivery sheath from guidewire 30, as shown in FIG. 12C.

Guidewire 30 may thereafter be used in a conventional rapid exchange manner for passing interventional devices, such as atherectomy devices, angioplasty device, and stent delivery systems, to desired locations in the vessel proximal to the location of vascular filter 20. Once the intended diagnostic or therapeutic treatment is performed, guidewire 30 is withdrawn proximally until the support hoop is drawn within the guidewire lumen of the interventional device, thereby closing the mouth of the filter and preventing emboli collected during the procedure from escaping into the patient's blood stream.

Advantageously, the vascular filter system, when used with delivery sheath 100, eliminates the need for a separate catheter exchange to insert a retrieval catheter to recover the filter. In addition, single-use delivery sheath 100 will discourage off-label repeat use of the vascular filter such as may occur if a separate delivery and retrieval sheath were used, because the delivery sheath is nonreusable once the filter has been deployed once. Further still, because delivery sheath 100 need not be capable of transmitting pushing forces, the walls of the sheath may be made very thin.

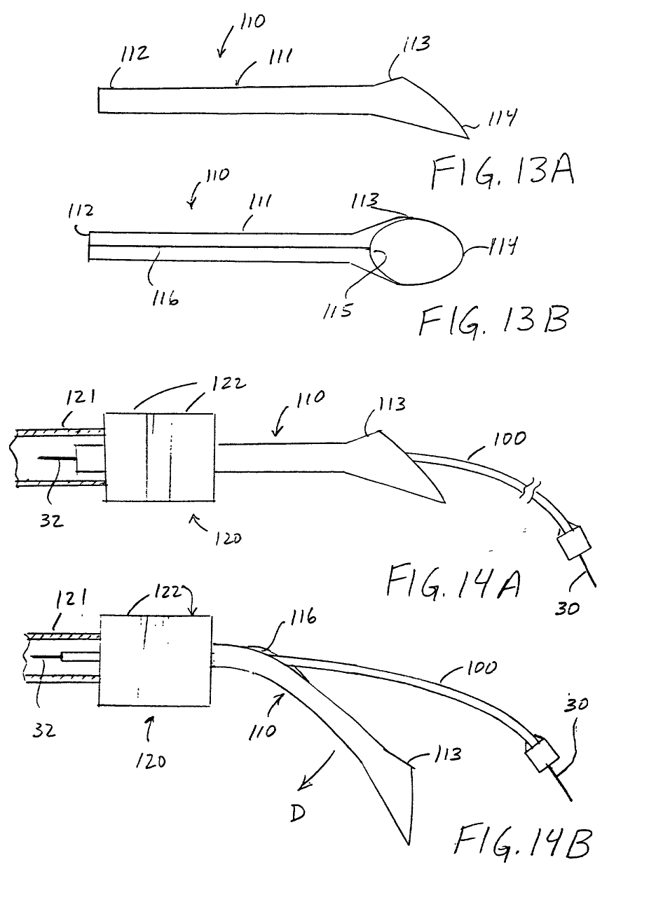

Referring now to FIGS. 13 and 14, introducer sheath 110 and methods of using that sheath in conjunction with vascular filter 20 and delivery sheath 100 of the present invention are described. Introducer sheath 110 is designed to pass floppy tip 32 of guidewire 30 through the rotating hemostatic valve of a guide catheter without kinking or tangling the floppy tip in the valve. Introducer sheath 110 comprises tubular body 111 having distal end 112, funnel-shaped proximal end 113, pull tab 114, central lumen 115 and full-length slit 116, and preferably comprises polyethylene, nylon or similar material, having sufficient rigidity to be pushed through a rotating hemostatic valve.

In a preferred method of use, illustrated in FIGS. 14A and 14B, introducer sheath 110 is advanced through rotating hemostatic valve 120 of guide catheter 121. As will of course be understood by one of skill in the art, guide catheter 121 may be a conventional multi-port guide catheter and includes a membrane that is selectively opened and sealed by rotating nuts 122 of the valve. Delivery sheath 100, which encloses vascular filter 20 and guidewire 30, then is inserted into funnel-shaped end 113 of the introducer sheath, and advanced to a location at which floppy tip 32 extends into guide catheter 121 distal to valve 120, as depicted in FIG. 14A.

Referring to FIG. 14B, pull tab 114 of introducer sheath 110 is pulled downward in the direction shown by arrow D so that delivery sheath 100 passes through slit 116 of the introducer sheath. Introducer sheath 110 is retracted proximally and peeled away from delivery sheath 100 as shown in FIG. 14B until the introducer sheath is entirely removed. Delivery sheath 100, vascular filter 20 and guidewire 30 then are advanced to the desired location in the vessel, and delivery sheath 100 is removed to deploy the vascular filter as described hereinabove with respect to FIGS. 12A-12C.

Advantageously, introducer sheath 110 permits the floppy tip 32 of guidewire 30 to be easily inserted through rotating hemostatic valve 120 of guide catheter 120. The peel-away operation of introducer sheath 110 facilitates rapid insertion of the vascular filter and guidewire into the guide catheter with little effort. In addition, slit 116 of introducer sheath 110 prevents destruction of the sheath after the single use, thus enabling the introducer sheath to be used to reintroduce the vascular filter in the same procedure. This may occur, for example, where the clinician begins inserting the vascular filter, but then needs to remove the filter and redirect the floppy tip during the same procedure.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. Apparatus suitable for filtering emboli comprising:
an elongated member having a distal region having a longitudinal axis;
a support hoop;
a suspension member coupling the support hoop to the distal region so that the support hoop may be eccentrically laterally displaced relative to the longitudinal axis; and
a blood permeable sac affixed to the support hoop so that the support hoop forms a mouth of the blood permeable sac.

2. The apparatus of claim 1, wherein the blood permeable sac comprises a biocompatible material.

3. The apparatus of claim 2, wherein the biocompatible material comprises a material chosen from a group consisting of polyethylene, polypropylene, polyurethane, polyester, polyethylene tetraphlalate, nylon and polytetrafluoroethylene.

4. The apparatus of claim 1, wherein the blood permeable sac comprises a woven material having a plurality of pores, the pores having a size determined by a weave pattern of the woven material.

5. The apparatus of claim 4, wherein each one of the plurality of pores has a diameter in a range of 20 to 400 microns.

6. The apparatus of claim 1, wherein the support hoop comprises a super-elastic material.

7. The apparatus of claim 1, wherein the support hoop comprises stainless steel.

8. The apparatus of claim 1, wherein the support hoop includes an articulation region.

9. The apparatus of claim 1, wherein the apparatus has a deployed state, wherein the support hoop engages an interior wall of a patient's vessel, and a delivery state, wherein the apparatus has a contracted configuration to permit insertion within a delivery sheath.

10. The apparatus of claim 1, further comprising a single-use delivery sheath.

11. The apparatus of claim 9, wherein the mouth of the blood permeable sac is closed when the apparatus is in the contracted configuration to prevent emboli from escaping from the blood permeable sac.

12. The apparatus of claim 11 wherein opposite sides of the first support hoop close towards one another when the apparatus is contracted to its contracted configuration.

13. The apparatus of claim 1, wherein the first support hoop comprises a radiopaque feature.

14. The apparatus of claim 1, further comprising a tube disposed in the distal region, and the suspension strut and blood permeable sac are affixed to the tube.

15. The apparatus of claim 14 wherein the distal region has a reduced diameter to accept the tube.

16. The apparatus of claim 1 wherein the suspension strut further includes one-or more side turns to stabilize and orient the apparatus in the deployed state.

17. The apparatus of claim 1 wherein the elongated member is a guide wire.

18. The apparatus of claim 14 further comprising a nose cone disposed on the tube.

19. The apparatus of claim 1, wherein the blood permeable sac has a length and a diameter that tapers along the length.

20. The apparatus of claim 1, wherein the blood permeable sac comprises a plurality of pores formed by laser drilling.

21. The apparatus of claim 1 further comprising a delivery sheath having a longitudinal perforation, the delivery sheath retaining the vascular filter in a contracted delivery state.

22. The apparatus of claim 21 further comprising an introducer sheath having a longitudinal slit, the introducer sheath facilitating insertion of the vascular filter into a guide catheter.

23. Apparatus suitable for filtering emboli comprising:
an elongated member having a distal region having a longitudinal axis;
a support hoop;
a suspension member coupling the support hoop to the distal region so that the support is disposed obliquely relative to the longitudinal axis; and
a blood permeable sac affixed to the support hoop so that the support hoop forms a mouth of the blood permeable sac.

24. The apparatus of claim 23, wherein the blood permeable sac comprises a biocompatible material.

25. The apparatus of claim 23, wherein the blood permeable sac comprises a plurality of pores having diameters in a range of 20 to 400 microns.

26. The apparatus of claim 23, wherein the support hoop includes an articulation region.

27. The apparatus of claim 23, wherein the apparatus has a deployed state, wherein the support hoop engages an interior wall of a patient's vessel, and a delivery state, wherein the apparatus has a contracted configuration to permit insertion within a delivery sheath.

28. The apparatus of claim 23, further comprising a single-use delivery sheath.

29. The apparatus of claim 23, wherein the mouth of the blood permeable sac is closed when the apparatus is in the contracted configuration to prevent emboli from escaping from the blood permeable sac.

30. The apparatus of claim 23, further comprising a tube disposed in the distal region, and the suspension strut and blood permeable sac are affixed to the tube.

31. The apparatus of claim 30 wherein the distal region has a reduced diameter to accept the tube.

32. The apparatus of claim 23 wherein the elongated member is a guide wire and the guide wire further comprises an articulation region.

33. A method of filtering emboli from flow within a vessel comprising:
providing a vascular filter mounted on a distal region of an elongated member, the elongated member having a longitudinal axis, the vascular filter comprising a support hoop, a suspension member coupling the support hoop to the distal region and a blood permeable sac affixed to the support hoop;
inserting the distal region of the elongated member and the vascular filter into a vessel;
deploying the vascular filter within the vessel at a location at which the support hoop is eccentrically laterally displaced relative to the longitudinal axis and the support hoop forms a mouth of the blood permeable sac.

34. The method of claim 33, wherein providing a vascular filter further comprises providing a vascular filter wherein the blood permeable sac comprises a biocompatible material.

35. The method of claim 33, wherein deploying the vascular filter further comprises articulating the support hoop at an articulation region.

36. The method of claim 33, wherein deploying the vascular filter comprises engaging the support hoop in apposition to an interior wall of the vessel.

37. The method of claim 33 further comprising, after completion of a diagnostic or therapeutic procedure, closing the mouth of the blood permeable sac and retrieving the vascular filter.

38. The method of claim 33 wherein inserting the distal region of the elongated member and the vascular filter into a vessel further comprises:
providing a guide catheter having a valve;
providing an introducer sheath;
inserting the introducer sheath into the guide catheter through the valve;
inserting the distal region of the elongated member and the vascular filter through the introducer sheath into the guide catheter; and
removing the introducer sheath.

39. The method of claim 33 wherein the introducer sheath includes a longitudinal slit and removing the introducer sheath further comprises removing the distal region of the elongated member and the vascular filter from the introducer sheath through the longitudinal slit.

40. The method of claim 33 wherein:
providing a vascular filter mounted on a distal region of an elongated member further comprises providing a vascular filter mounted on a distal region of an elongated member enclosed within a delivery sheath, the delivery sheath having a longitudinal perforation; and
inserting the distal region of the elongated member and the vascular filter into a vessel further comprises advancing the distal region of the elongated member and the vascular filter to a desired location in the vessel, and then holding the elongated member stationary while retracting the delivery sheath.
